(19) United States
(12) Patent Application Publication
(10) Pub. No.: US 2025/0312480 A1
(43) Pub. Date: Oct. 9, 2025

(54) PLATFORM OF DRUG DELIVERY BASED ON SELF-LYSIS OF ECN, CONSTRUCTING METHOD AND USE OF THE SAME

(71) Applicant: Jiangxi Normal University, Nanchang City (CN)

(72) Inventors: Jiaofang HUANG, Nanchang City (CN); Ning JIANG, Nanchang City (CN)

(73) Assignee: Jiangxi Normal University, Nanchang City (CN)

(21) Appl. No.: 19/015,767

(22) Filed: Jan. 10, 2025

(30) Foreign Application Priority Data
Apr. 7, 2024 (CN) 202410409102.2

(51) Int. Cl. A61K 47/69; A61K 31/415; A61K 31/704; A61K 47/54; A61P 35/00; C12N 15/70; C12R 1/19

(52) U.S. Cl. CPC A61K 47/6901; A61K 31/415; A61K 31/704; A61K 47/542; A61K 47/6911; A61P 35/00; C12N 15/70; C12R 2001/19

(57) ABSTRACT

A platform of drug delivery based on self-lysis of ECN, a constructing method, and use of the platform are provided. The platform of drug delivery comprises ECN, a Pvhb-GST-PhiX174E circuit genetically introduced into ECN, and a drug encapsulation material. When the platform is used as an anti-cancer drug carrier, the platform delivers a high concentration of chemotherapeutic drugs to a tumor site. Additionally, through the Pvhb-GST-PhiX174E circuit, ECN is induced to lyse in response to tumor microenvironment signals, leading to cell death in the tumor region and enhancing ECN's biosafety in vivo. Furthermore, ECN within the platform of drug delivery based on self-lysis of ECN serves as an immune adjuvant, stimulates the immune cell activity in the tumor site, triggers acute inflammation, and strengthens the anti-tumor effect, thereby achieving multi-modal anti-tumor therapy through live-cell, chemotherapy, and immunotherapy.

This application claims the benefit of priority of Chinese Patent Application No. 202410409102.2 filed on Apr. 7, 2024, the contents of which are incorporated by reference as if fully set forth herein in their entirety.

### FIELD OF INVENTION

The present disclosure relates to a field of biomedicine, specifically to a platform of drug delivery based on self-lysis of Escherichia coli Nissle 1917 (ECN), a constructing method and use of the same.

### BACKGROUND OF INVENTION

Cancer is one of the primary health threats today. With lifestyle changes in recent years, cancer incidence has been trending younger. Colon cancer, as a malignant cancer, poses a serious threat to human life and has led to significant medical costs. Traditional cancer treatments have shown limited effectiveness. For example, surgery can remove the bulk of tumor cells but relies heavily on tumor imaging and is only effective for early-stage tumors. While radiotherapy and chemotherapy can be used alongside surgery, their severe side effects cause immense suffering for patients. One major challenge in clinical oncology is tumor resistance to chemotherapy drugs, especially with prolonged use.

In vivo drug treatment strategies offer new approaches for cancer therapy. Escherichia coli Nissle 1917 (ECN) is a facultative anaerobic probiotic and a non-pathogenic E. coli strain. Due to the hypoxic microenvironment of tumors, ECN can specifically target tumor sites and colonize the hypoxic core. This enables ECN not only to compete with tumor cells for nutrients, inhibiting their growth, but also to kill them through its metabolic byproducts, making it a promising candidate for tumor therapy. However, ensuring biosafety for in vivo applications remains a major concern.

### SUMMARY OF DISCLOSURE

To address the technical challenges in the background, the present disclosure leverages the advantage of ECN's active tumor targeting. The objective of the present disclosure is to provide a platform of drug delivery based on self-lysis of ECN, a constructing method of the platform and use of the platform.

To achieve the above objective, the present disclosure provides the following technical solutions:

A first aspect of the present disclosure provides a platform of drug delivery based on self-lysis of ECN, which includes an Escherichia coli Nissle 1917 cell (ECN); a lysis circuit, including Promoter of Vitreoscilla Hemoglobin gene, Glutathione S transferase, and PhiX174E (Pvhb-GST-PhiX174E), was genetically introduced into the ECN for automatically controlling release of a drug from the ECN at tumor sites; and a drug encapsulation material for anchoring the drug onto a surface of the ECN, wherein the drug encapsulation material is selected from the group consisting of liposomes (Lipo) or tannic acid (TA).

We first encapsulate nanomaterials with tumor acidic microenvironment degradation functions, such as Lipo or TA, on the surface of ECN. When this drug reaches the tumor site, the acidic tumor microenvironment can degrade these materials, thereby releasing the drug. Considering the in vivo safety of ECN, we have successfully constructed and introduced a lysis circuit responsive to the tumor microenvironment into ECN. This circuit can specifically lyse ECN in response to the tumor hypoxic microenvironment. At the same time, the lytic products can act as immune adjuvants to stimulate macrophages, achieving a combined chemotherapy-immunotherapy strategy.

Preferably, the TA and Lipo were riveted on the surface of ECN.

Preferably, the Lipo are negatively charged Lipos. TA chelates with iron ions to form a viscous complex, which is then encapsulated on the surface of ECN.

Preferably, the Pvhb-GST-PhiX174E circuit is hypoxia-inducible in a tumor microenvironment, including a hypoxia-inducible promoter (Pvhb), a GST tag, and phiX174E lysis proteins.

Preferably, the platform of drug delivery has a diameter ranging from 800 to 1500 nm.

Preferably, compared to the wild type ECN, the size of the drug delivery platform after encapsulation increased by 10 nm to 200 nm.

A second aspect of the present disclosure provides a constructing method of the abovementioned platform of drug delivery based on self-lysis of ECN, comprising the following steps of:
S1: constructing a lysis plasmid containing the pGEX-Pvhb-GST-PhiX174E circuit;
S2: introducing the lysis plasmid containing the pGEX-Pvhb-GST-PhiX174E circuit into ECN competent cells or integrating a fragment of the Pvhb-GST-PhiX174E circuit into a genome to obtain recombinant ECN engineered bacteria;
S3: adding the obtained recombinant ECN engineered bacteria into deionized water to prepare a bacterial solution of the recombinant ECN engineered bacteria;
S4: sequentially adding a TA solution, duoroubixing (DOX), and a FeCl₃ solution into the bacterial solution and conducting vortex to form the platform of drug delivery based on self-lysis of ECN;

Alternatively, S3′: adding the recombinant ECN engineered bacteria obtained from the step S2 into a PBS solution to prepare a PBS solution of ECN engineered bacteria;
S4′: adding a glycol chitosan solution into the obtained PBS solution of ECN engineered bacteria, then stirring, and rinsing with PBS to obtain a mixed solution of ECN engineered bacteria;
S5′: dissolving soybean lecithin and cholesterol in ethanol, then adding a solution containing Celecoxib (CXB), stirring, and performing ultrasonication and ultrafiltration concentration to obtain a CXB@Lipo (C@L) solution; and
S6′: adding the C@L solution into the mixed solution of ECN engineered bacteria obtained from the step S4′, stirring, performing centrifuge, collecting the precipitate, and rinsing with PBS to obtain the platform of drug delivery based on self-lysis of ECN.

Preferably, in the step S3, a density of the bacterial solution of the recombinant ECN engineered bacteria in the step S3 is 10\hat{ }6-10\hat{ }8 cfu/ml.

Preferably, in the step S4, a ratio of the TA solution, DOX, and the FeCl3 solution in the step S4 is 10-15 nM:1-2 mg:10-15 nM, a concentration of the TA solution is 5-15 mM and a concentration of the FeCl3 solution is 5-15 mM; and a time period of conducting vortex is 3-10 min.

Preferably, in the step S3′, a density of the PBS solution of ECN engineered bacteria in the step S3′ is 10\hat{ }6-10\hat{ }8 cfu/ml.

Preferably, in the step S4′, an amount of the glycol chitosan solution is 2-4 mg, a stirring speed is 220 rpm, and a time period of stirring is 30 min.

Preferably, in the step S5′, a molar ratio of the soybean lecithin to the cholesterol is 4:1, a concentration of the C@L solution is 2 mg/ml, a stirring speed is 400 rpm, and a time period of stirring is 5 min.

Preferably, in the step S6′, a speed of rinsing with PBS is 3500 rpm and a time period of rinsing with PBS is 5 min.

A third aspect of the present disclosure provides a method for treating a tumor-related disease in a subject in need thereof, including a step of administrating to the subject the above platform of drug delivery based on self-lysis of ECN.

The present disclosure offers the following beneficial effects:
(1) The present disclosure constructs a platform of drug delivery based on self-lysis of ECN, which includes an Escherichia coli Nissle 1917 cell (ECN), a Pvhb-GST-PhiX174E circuit introduced into ECN through genetic engineering, and a drug encapsulation material anchored to the surface of the ECN. Experimental results show that when this platform of drug delivery serves as an anti-cancer drug carrier, it not only loads a large amount of chemotherapy drugs to reach the tumor site-featuring a high drug-loading rate (up to 70%)—but also induces ECN lysis at the tumor site through the lysis circuit Pvhb-GST-PhiX174E in response to tumor microenvironment signals (e.g., tumor hypoxia). This smart release does not affect the activity of ECN, so that the biosafety of ECN in vivo is enhanced.
(2) The ECN in the platform of drug delivery based on self-lysis of ECN constructed by the present disclosure can also act as an immunoadjuvant to stimulate immune cell activity at the tumor site, trigger acute inflammation, and enhance anti-tumor effects, thereby achieving a combination of in vivo-chemotherapy-immunotherapy for multimodal anti-tumor treatment. Therefore, the platform has promising application potential.

### BRIEF DESCRIPTION OF DRAWINGS

To more clearly illustrate the technical solutions in the embodiments of the present disclosure, a brief introduction of the drawings described in the embodiments is provided below. It is apparent that the following drawings are merely some embodiments of the present disclosure, and additional drawings would be obtained by those skilled in the art based on these drawings without inventive effort.

FIG. 1: Plasmid map of constructing the pGEX-Pvhb-GST-PhiX174E circuit.

FIG. 2: TEM images of the platform of drug delivery based on self-lysis of ECN prepared in Examples 1-2.

FIG. 3: Co-localization effect images of ECN, Duoroubixing@Tannic acid (D@T) and Celecoxib@Lipo (C@L) when using the platform of drug delivery based on self-lysis of ECN prepared in Example 1-2.

FIG. 4: Loading efficiency results of Examples 1-2 at different concentrations on the surface of ECN.

FIG. 5: Growth curves of ECN before and after drug loading of Example 1-2.

FIG. 6: Promoter-induced expression of fluorescent protein under hypoxic conditions.

FIG. 7: Lysis effect of recombinant ECN under hypoxic conditions.

FIG. 8: Inhibition effect on COX-2 expression in tumor cells.

FIG. 9: Results of inhibiting the growth of colon cancer cells in vitro (Examples 1-2).

FIG. 10: Results of inhibiting tumor growth of Examples 1-2 in a mouse model.

FIG. 11: In vivo induction of macrophage polarization from M2 to M1 type.

FIG. 12: Induction of immune cell infiltration results.

FIG. 13: Apoptosis index results for tumor cells in vivo.

FIG. 14: In vivo safety evaluation results.

### DETAILED DESCRIPTION OF PREFERRED EMBODIMENTS

The following description provides specific details, such as particular system configurations and techniques, which are used for explaining but not for limiting, to thoroughly understand the embodiments of the present disclosure. However, those skilled in the art should understand that other embodiments of the disclosure may be practiced without these specific details.

### Example 1

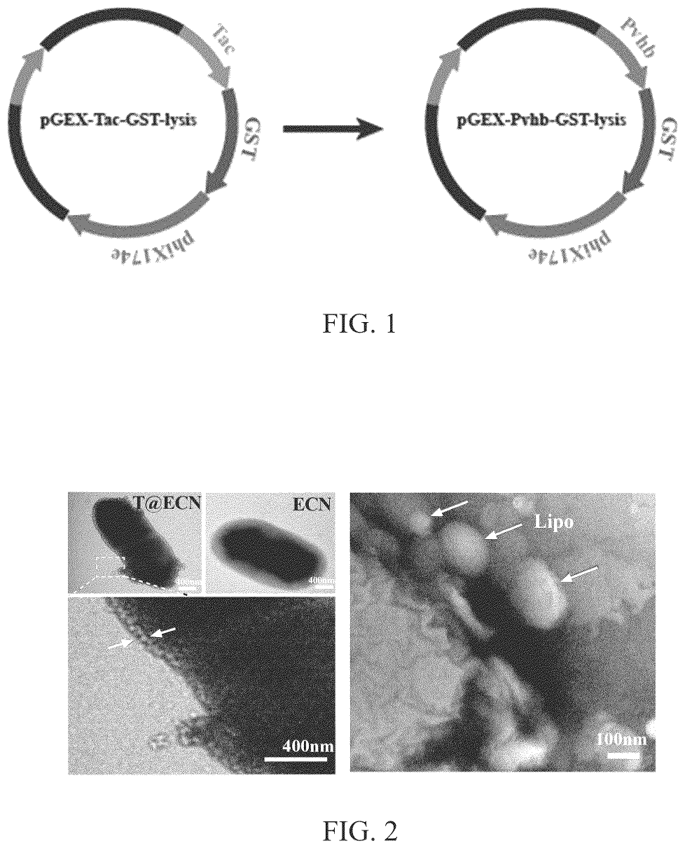

A constructing method of a platform of drug delivery based on self-lysis of ECN, comprising the following steps:
S1: Referring to FIG. 1, constructing the Pvhb-GST-PhiX174E circuit by creating a lysis plasmid containing pGEX-Pvhb-GST-PhiX174E. The specific steps are as follows:
(1) Construction of the lysis plasmid pGEX-Tac-GST-PhiX174E:
(a) The pGEX plasmid was served as an expression vector. Performing enzyme digestion on the pGEX plasmid by using EcoR1 and BamH1. The enzyme digestion system consists of pGEX (2 μg, 20 μl), EcoR1 (1.5 μg), BamH1 (1.5 μg), Buffer (3 μg), and H₂O (4 μg), which incubate at 37° C. for 1 hour.
(b) After enzyme digestion, performing electrophoresis on a 1% agarose gel under the following conditions: 110 mV for 25 minutes. Next, using a TAKARA gel recovery kit to retrieve the linear fragment and quantify with a Nanodrop.
(c) PCR parameters were set according to the PrimaStart MAX manual to amplify the recovered linear fragment. The PCR system was as follows: PhiX174E gene (1 μl), Primer-1 (1 μl), Primer-2 (1 μl), PrimaStart MAX (10 μl), and H₂O (7 μl). Conducting PCR amplification at 72° C. for 40 minutes. After completing the PCR amplification, using 1% agarose gel electrophoresis to collect a 230 bp nucleic acid fragment for use later.
(d) Using the Seamless Cloning Kit from Vazyme Biotech to seamlessly clone the lysis fragment into the pGEX backbone. The ligation system includes linear pGEX (50 ng), lysis (10 ng), and Seamless Cloning (5 μl). Incubation was performed at 50° C. for 15 minutes as per the instructions.
(e) After incubation, transferring the cultures into DH5α competent cells. Ice-bath was performed for 30 minutes, and then heat-shock was performed at 42° C. for 90 seconds. 100 μl product was taken out and plated on a Carb 50 plate.
(f) After 12 hours of plating, picking a single colony and culturing it. Using PrimaStart MAX for PCR, with the following PCR conditions: Bacterial culture (1 μl), Primer-1 (0.5 μl), Primer-2 (0.5 μl), PrimaStart MAX (5 μl), and H₂O (3 μl). Amplification was performed at 72° C. for 40 minutes to obtain the lysis plasmid of pGEX-Tac-GST-PhiX174E.
(2) Promoter Replacement:
(g) Primers were designed to amplify the backbone of the lysis plasmid of pGEX-Tac-GST-PhiX174E by PCR. The PCR amplification system is as follows: pGEX-GST-PhiX174E (1 μl), Primer-3 (1 μl), Primer-4 (1 μl), PrimaStart MAX (10 μl), and H₂O (7 μl). After PCR amplification, performing electrophoresis on a 1% agarose gel, collecting the 6000 bp band, and recovering the gel by using a gel recovery kit to obtain the linearized vector pGEX-PhiX174E without the Tac promoter.
(h) the Pvhb promoter sequence was synthesized through Qingke Biotechnology, and the Pvhb promoter was connected to pGEX-GST-PhiX174E by using seamless cloning. The ligation system includes linear pGEX-GST-PhiX174E, Pvhb, and Seamless Cloning, and reacts at 50° C. for 15 minutes.
(i) After completing the reaction, transferring the plasmid into DH5α strain for amplification. Finally, transferring the verified correct lysis plasmid, containing pGEX-Pvhb-GST-PhiX174E, into competent ECN cells for use later.
S2: Introducing the obtained lysis plasmid into competent ECN cells to obtain the recombinant ECN-PL engineered strain.
S3: Adding a certain amount of the recombinant ECN-PL engineered strain into deionized water to prepare a solution with a concentration of 10\hat{ }8 cfu/ml. Next, adding 15 nM TA solution (at a concentration of 10 mM) and performing vortex for 1 minute to obtain Bacterial Solution 1.
S4: Adding DOX (1 mg) into the Bacterial Solution 1 and performing vortex for 2 minutes to obtain Bacterial Solution 2.
S5: Adding 10 nM ferric chloride (FeCl₃) solution (at a concentration of 15 mM) into the Bacterial Solution 2, and then performing vortex for 2 minutes, and rinsing with PBS three times to obtain the self-lysing platform of drug delivery, referred to as Duoroubixing@Tannic acid@Escherichia coli Nissle 1917 (DOX@TA@ECN), abbreviated as D@T@ECN.

### Example 2

A1: Constructing a lysis plasmid containing pGEX-Pvhb-GST-lysis (same as Example 1), and introducing the plasmid into competent ECN cells, or integrate the Pvhb-GST-PhiX174E fragment into the ECN genome to obtain the recombinant ECN engineered bacteria.
A2: Adding the obtained recombinant ECN bacteria to a PBS solution at a concentration of 10\hat{ }7 cfu/ml, and then adding 2 mg of ethylene glycol chitosan solution, and stirring at 220 rpm for 30 minutes.
A3: Dissolving soybean lecithin and cholesterol in ethanol at a molar ratio of 4:1. Slowly adding this mixture into a CXB-containing solution at 400 rpm, and stirring for 5 minutes to obtain the Celecoxib@Lipo solution, referred to as C@L.
A4: Sonicating the solution from the step A3 for 10 minutes, and then concentrating the solution with a 10 kDa ultrafiltration centrifuge tube, rinsing with PBS three times. The concentration of C@L is 1 mg/ml.
A5: Adding the solution from the step A4 into the solution in the step A2, and keeping stirring for 30 minutes, and performing centrifuge to collect the precipitate. The precipitate was rinsed with PBS three times at 3500 rpm for 5 minutes to obtain the self-lysing platform of drug delivery, referred to as CXB@Lipo@ECN-PVHB-LYSIS, abbreviated as C@L@ECN-PL.

### Example 3

Similar to Example 2, except that the concentration of C@L is 1 mg/ml in this example.

### Example 4

Similar to Example 2, except that the concentration of C@L is 2 mg/ml in this example.

Transmission electron microscopy was used to characterize the morphology of the self-lysing platform of drug delivery based on recombinant engineered ECN bacteria produced in Examples 1 and 2. Results are shown in FIG. 2.

From FIG. 2, it can be observed that the ECN surface in the self-lysing platform of drug delivery produced in Example 1 becomes rough after being encapsulated with TA, indicating that TA successfully covers on the ECN surface. In the self-lysing platform of drug delivery produced in Example 2, D@L is uniformly adsorbed on the ECN surface, showing that the D@L@ECN is successfully prepared.

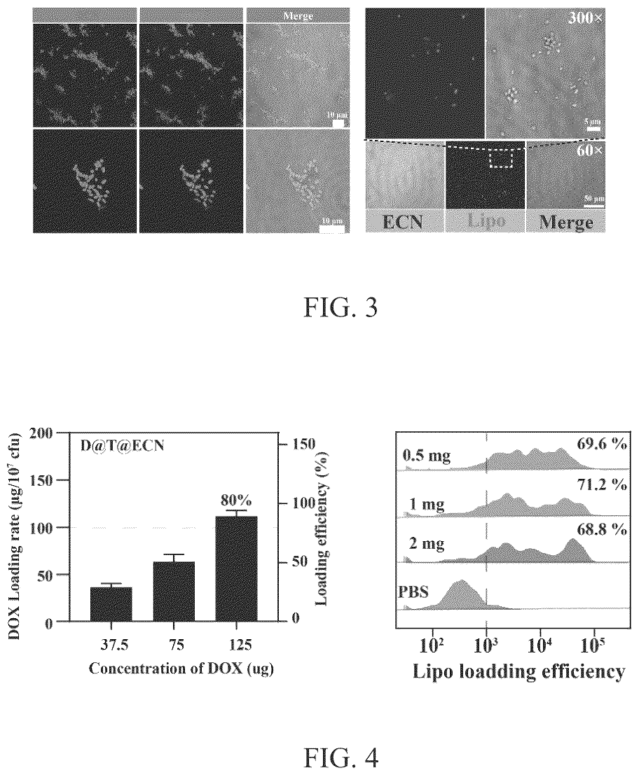

A confocal laser scanning microscope was used to co-localize the drug (D@T) and ECN in the self-lysing platform of drug delivery from Example 2. First, the lipophilic dye DIO was used to stain D@T, as DIO would bind to TA and emit green fluorescence. Next, D@T@ECN was observed under a confocal microscope, with results as shown in FIG. 3.

As shown in FIG. 3, C@L emits green fluorescence under 488 nm excitation, which overlaps with ECN (Merge), indicating successful loading of D@T@ECN on the ECN surface.

4. D@T@ECN was mixture with different concentration of DOX. The maximum adsorption efficiency of C@L@ECN was evaluated by using flow cytometry. 10\hat{ }7 cfu of ECN was used herein to adsorb different concentrations (0 mg, 0.5 mg, 1 mg, 2 mg) of C@L. Results are shown in FIG. 4.

From FIG. 4, D@T@ECN loading effective of DOX was 80% at DOC concentration at 125 μg/μl. C@L@ECN can be noted that the adsorption efficiency of ECN is 69.6% at 0.5 mg C@L, and the maximum adsorption efficiency is 71.2% at 1 mg C@L, indicating that a maximum adsorption rate is 71.2% for this platform of drug delivery.

5. Analysis of ECN activity (growth status) after drug loading was performed.

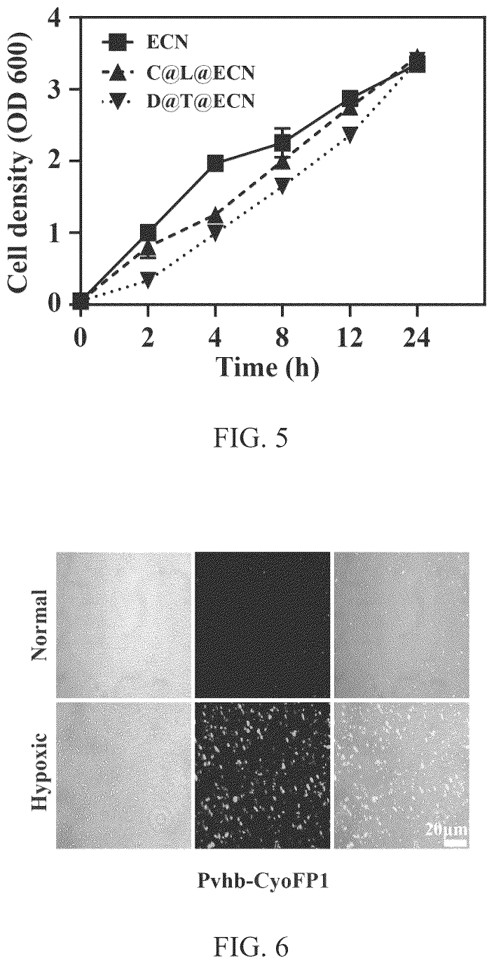

10\hat{ }3 cfu of ECN, D@T@ECN, and Celecoxib@Lipo@Escherichia coli Nissle 1917 (C@L@ECN) were inoculated into 3 ml LB medium, respectively, incubated at 220 rpm and 37° C., and sampled at different time points. The concentration of bacterial solution was measured at 600 nm by using a UV-Vis spectrophotometer, with results as shown in FIG. 5.

From FIG. 5, it can be noted that the drug loading has no significant impact on ECN growth.

6. Induction of fluorescent protein expression under hypoxic conditions

CyoFP1 fluorescent protein was used as a reporter gene to test the hypoxia-inducible circuit. 10\hat{ }3 cfu of ECN was inoculated into LB medium and incubated at 220 rpm and 37° C. When the OD600 of the bacterial solution reached 0.4, the culture dishes were sealed with paraffin to isolate oxygen, and incubation continued for 3 hours. Observations were then made by using a confocal laser scanning microscope, with results as shown in FIG. 6. FIG. 6 shows that the ECN can be induced to express the target protein under hypoxic conditions.

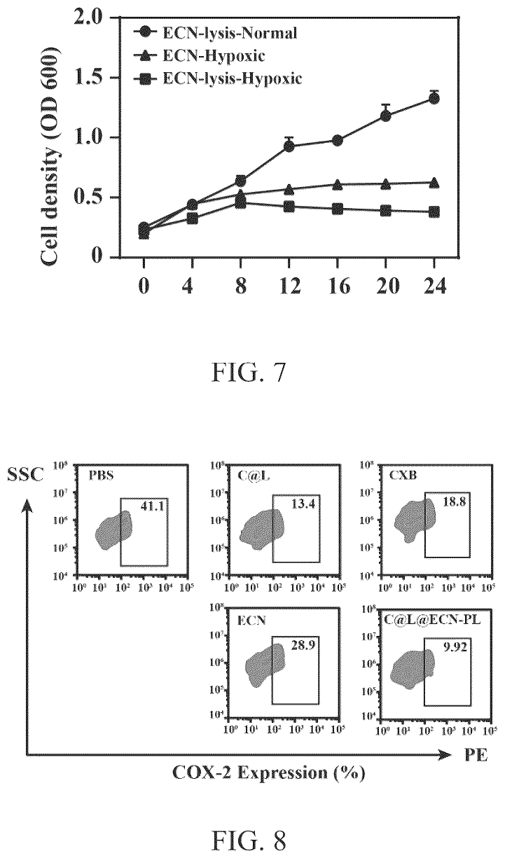

7. Verification of Pvhb-GST-PhiX174E circuit expression in ECN under controlled oxygen levels in LB medium. Results are shown in FIG. 7.

FIG. 7 shows that Pvhb-GST-PhiX174E is induced under hypoxic conditions, resulting in ECN lysis, thereby enabling control of ECN cell numbers.

8. Study on the inhibition of COX-2 expression in tumor cells.

This platform of drug delivery inhibits COX-2 expression in tumor cells, thereby reducing tumor cell anti-apoptotic ability and enhancing the anti-tumor effects of chemotherapy drugs.

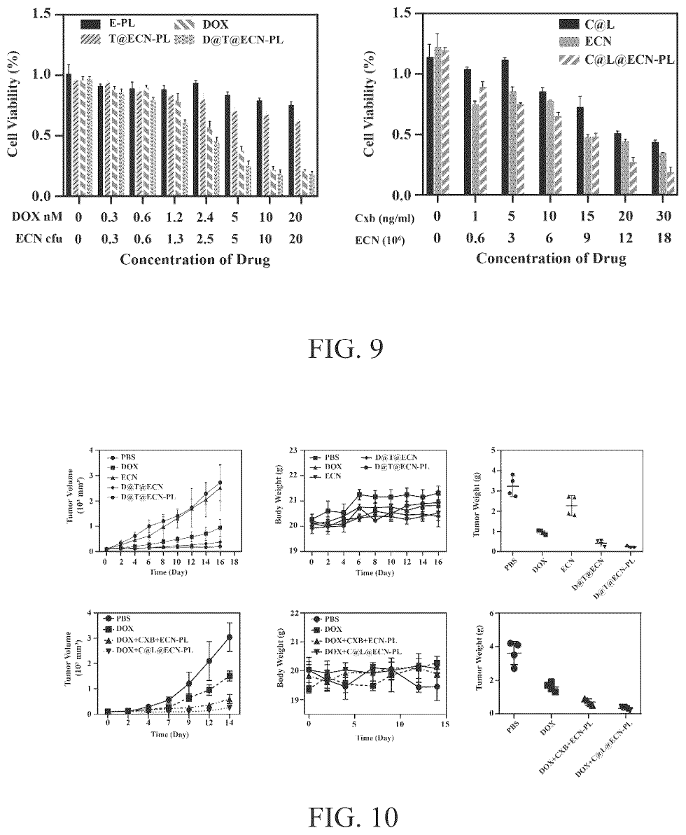

9. As Examples 1-2, toxicity test of the platform of drug delivery on CT26 colon cancer cells. Results are shown in FIG. 9.

FIG. 9 shows that tumor cell viability decreases as drug concentration increases, which indicates that the platform of drug delivery effectively inhibits tumor cell growth.

10. Anti-tumor efficacy test of the platform of drug delivery produced in Example 2 on a subcutaneous CT26 mouse tumor model

In a subcutaneous transplantation tumor model, the experiment started when mouse tumor volume reached 100 mm\hat{ }3. Mice were randomly divided into four groups with each group including five mice, following that the drug (10\hat{ }7 cfu per mouse) was injected via the tail vein. Tumor volume was measured every two days by using the formula: A×B2/2A×B2/2 (A: measured value of long diameter of tumor volume, B: measured value of short diameter of tumor volume). Results are shown in FIG. 10.

FIG. 10 shows that tumor growth in the mice was inhibited by 80% after 14 days of treatment, with no significant changes in body weight, which demonstrates significant in vivo anti-tumor efficacy of the platform of drug delivery.

11. In vivo study on macrophage polarization to M1 type.

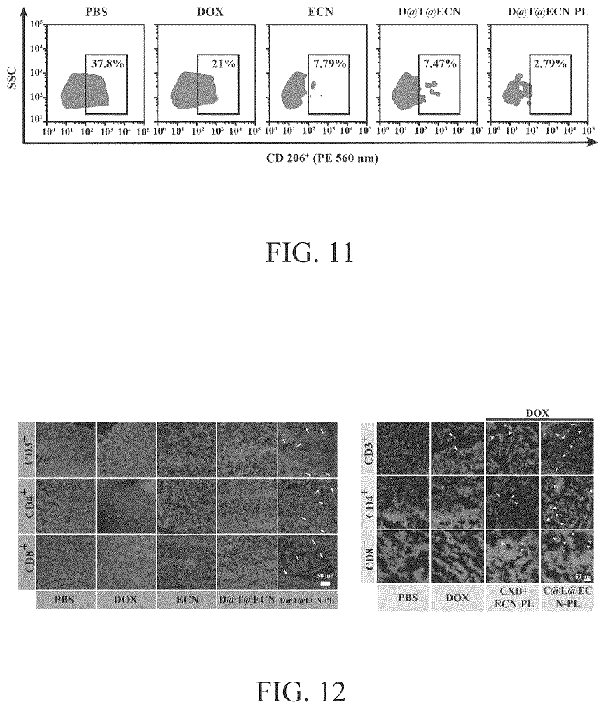

FIG. 11 shows that ECN lysis in the tumor area stimulates immune cell polarization, which induces macrophage polarization to M1 type so as to enhance immune anti-tumor effects.

12. Evaluation of immune cell infiltration induction.

After 14 days of treatment, tumor cells from mice were collected and sectioned (6 μm thick) by using a cryostat. Immune markers were stained, and the results are shown in FIG. 12.

FIG. 12 shows enhanced infiltration of CD3⁺, CD4⁺, and CD8⁺T lymphocytes in the tumor area of the mice treated with the platform of drug delivery based on self-lysis of ECN.

13. Evaluation of Tumor Cell Apoptosis Indicators in Vivo.

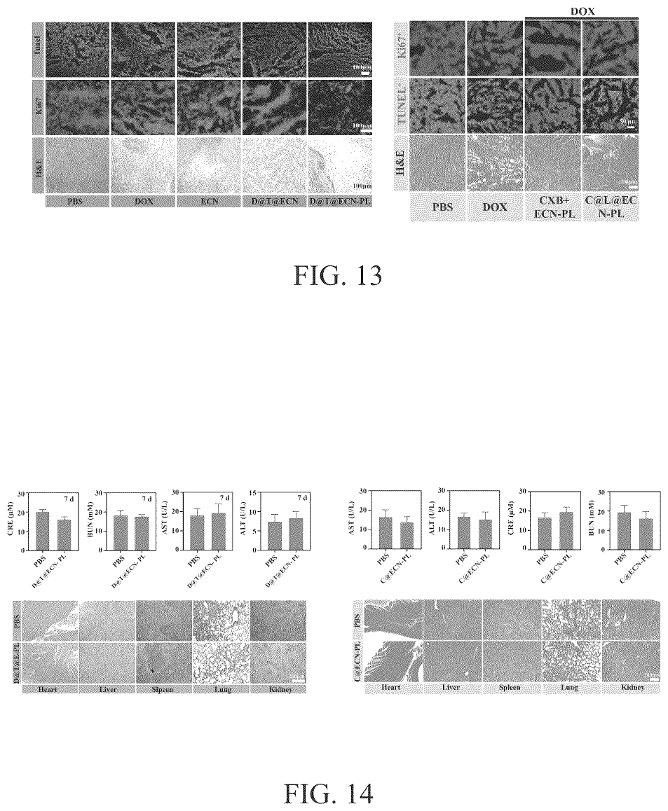

Tumor cell proliferation (KI67) and apoptosis indicators (TUNEL) were assessed by using frozen sections with the results as shown in FIG. 13.

FIG. 13 shows that tumor cell proliferation was inhibited after treatment with the platform of drug delivery based on self-lysis of ECN, and a significant induction of tumor cell apoptosis occurred. This result indicates that the platform of drug delivery based on self-lysis of ECN effectively inhibits tumor cell growth.

14. In Vivo Safety Evaluation

Blood from treated mice was tested, with results shown in FIG. 14.

FIG. 14 shows that after 14 days of treatment, there were no significant changes in function indicators for liver and kidney of the mice when compared to the PBS group. This result demonstrates that the platform of drug delivery based on self-lysis of ECN is safe and effective.

In summary, the present disclosure has developed a high-efficiency and safe self-lysing drug delivery platform based on a recombinant ECN. This platform not only loads a large amount of (chemotherapy) drugs to reach tumor sites but also responds to tumor microenvironment signals for smart drug release at the tumor sites, thereby enhancing the biosafety of ECN in vivo.

The present disclosure is not limited to the specific embodiments described above. Variations made by those skilled in the art based on the above concept, without creative labor, are within the protection scope of the present disclosure.

What is claimed is:

1. A platform of drug delivery based on self-lysis of ECN, comprising an Escherichia coli Nissle 1917 cells (ECN), a Pvhb-GST-PhiX174E circuit genetically introduced into the ECN for automatically controlling release of a drug from the ECN at a tumor site, and a drug encapsulation material for anchoring the drug onto a surface of the ECN, wherein the drug encapsulation material is selected from the group consisting of liposomes (Lipo) and tannic acid.

2. The platform of drug delivery based on self-lysis of ECN according to claim 1, wherein the tannic acid and the Lipo were riveted on the surface of ECN.

3. The platform of drug delivery based on self-lysis of ECN according to claim 1, wherein the Pvhb-GST-PhiX174E circuit is hypoxia-inducible in a tumor microenvironment, including a hypoxia-inducible promoter (Pvhb), a GST tag, and phiX174E lysis proteins.

4. The platform of drug delivery based on self-lysis of ECN according to claim 1, wherein the platform of drug delivery has a diameter ranging from 800 to 1500 nm, and the size of ECN increased by 10-200 nm after encapsulation with the tannic acid or the Lipo.

5. A constructing method of the platform of drug delivery based on self-lysis of ECN according to any of claims 1-4, comprising the following steps of:
S1: constructing a lysis plasmid containing the pGEX-Pvhb-GST-PhiX174E circuit;
S2: introducing the lysis plasmid containing the pGEX-Pvhb-GST-PhiX174E circuit into ECN competent cells or integrating a fragment of the Pvhb-GST-PhiX174E circuit into a genome to obtain recombinant ECN engineered bacteria;
S3: adding the recombinant ECN engineered bacteria into deionized water to prepare a bacterial solution of the recombinant ECN engineered bacteria; and
S4: sequentially adding a TA solution, duoroubixing (DOX), and a FeCl₃ solution into the bacterial solution and conducting vortex to form the platform of drug delivery based on self-lysis of ECN; or
performing the following steps after the step S2,
S3′: adding the recombinant ECN engineered bacteria obtained from the step S2 into a PBS solution to prepare a PBS solution of ECN engineered bacteria;
S4′: adding a glycol chitosan solution into the PBS solution of ECN engineered bacteria, then stirring and rinsing with PBS to obtain a mixed solution of ECN engineered bacteria;
S5′: dissolving soybean lecithin and cholesterol in ethanol, then adding a solution containing Celecoxib (CXB), stirring, and performing ultrasonication and ultrafiltration concentration to obtain a CXB@Lipo solution; and
S6′: adding the CXB@Lipo solution into the mixed solution of ECN engineered bacteria obtained from the step S4′, stirring, performing centrifuge, collecting the precipitate, and rinsing with PBS to obtain the platform of drug delivery based on self-lysis of ECN.

6. The constructing method according to claim 5, wherein a density of the bacterial solution of the recombinant ECN engineered bacteria in the step S3 is 10\hat{ }6-10\hat{ }8 cfu/ml.

7. The constructing method according to claim 5, wherein a ratio of the TA solution, DOX, and the FeCl3 solution in the step S4 is 10-15 nM: 1-2 mg: 10-15 nM, a concentration of the TA solution is 5-15 mM and a concentration of the FeCl3 solution is 5-15 mM; and a time period of conducting vortex is 3-10 min.

8. The constructing method according to claim 5, wherein a density of the PBS solution of ECN engineered bacteria in the step S3′ is 10\hat{ }6-10\hat{ }8 cfu/ml.

9. The constructing method according to claim 5, wherein:
in the step S4′, an amount of the glycol chitosan solution is 2-4 mg, a stirring speed is 220 rpm, and a time period of stirring is 30 min;
in the step S5′, a molar ratio of the soybean lecithin to the cholesterol is 4:1, a concentration of the CXB@Lipo solution is 0.5-2 mg/ml, a stirring speed is 400 rpm, and a time period of stirring is 5 min; and
in the step S6′, a speed of rinsing with PBS is 3500 rpm and a time period of rinsing with PBS is 5 min.

10. A method for treating a tumor-related disease in a subject in need thereof, comprising a step of administering to the subject the platform of drug delivery based on self-lysis of ECN according to claim 1, thereby treating the tumor-related disease.
